(19) United States
(12) Patent Application Publication
(10) Pub. No.: US 2025/0302868 A1
(43) Pub. Date: Oct. 2, 2025

(54) HYPOCHLOROUS ACID-BASED DRY EYE FORMULATIONS

(71) Applicant: OCuSOFT, Inc., Rosenberg, TX (US)

(72) Inventors: Paramita Sarkar, Rosenberg, TX (US); Brandon Sands, Hungerford, TX (US)

(21) Appl. No.: 18/617,328

(22) Filed: Mar. 26, 2024

(51) Int. Cl. A61K 31/79; A61F 9/00; A61K 9/00; A61K 47/02

(52) U.S. Cl. CPC A61K 31/79; A61F 9/0008; A61K 9/0048; A61K 47/02

(57) ABSTRACT

A dry eye composition containing hypochlorous acid and a demulcent. The demulcent can be polyvinylpyrrolidoe with a molecular weight of 30-50 kDa. The composition can be applied to the surface of the eye with a dropper device or a spray dispenser. Critically, hypochlorous acid is stable in the dry eye composition and maintains its effectiveness at preventing microbial growth.

### FIELD OF THE INVENTION

The present invention relates generally to dry eye formulations and methods for using such formulations.

### BACKGROUND

Ocular health encompasses the well-being of the eyes and related structures, including the eyelids and tear film. Within the eyelids, there are crucial lacrimal and meibomian glands working in tandem to produce layers of tear film vital for maintaining healthy eyes. Every time a person blinks, a fresh tear film forms, gently spreading across the cornea to lubricate the eye's surface and naturally clear away foreign particles. In addition to its protective function, the tear film also plays a pivotal role in preserving eye moisture, optimizing ocular homeostasis, and enhancing overall ocular health.

Dry eye occurs when there is insufficient tear production to keep the eyes adequately moist or when the tear film evaporates rapidly from the eye's surface. People over the age of 50, those with autoimmune conditions, and contact lens wearers are particularly susceptible to experiencing dry eye. Additionally, an inadequate diet or specific environmental factors can contribute to dryness in the eyes. Common symptoms associated with dry eye include redness, blurred vision, stinging, burning, a gritty feeling, and sensitivity to light. Consequently, dry eye has the potential to lead to vision problems and can have a negative impact on overall ocular health.

Dry eye can be linked to various complications, including infection, inflammation, and damage to the corneal surface. In the absence of the protective tear film on the eye's surface, otherwise harmless bacteria can lead to infections in the eye margins. Both bacterial infections and untreated dry eye can result in inflammation in the ocular region, worsening the dryness. Moreover, prolonged dry eye can lead to abrasions on the eye's surface and, in severe cases, permanent damage and vision loss.

Typical treatments for dry eye encompass various approaches, including dietary adjustments, increased hydration, avoidance of adverse environmental conditions, over-the-counter eye drops or artificial tears, prescription medications, punctum plugs, and blepharoplasty. In many cases, over-the-counter eye drops prove effective in alleviating the discomfort associated with dry eye and reducing the risk of bacterial infections. These eye drops primarily contain compounds known as demulcents, which create a protective film over the eye's surface, slowing the evaporation of the existing tear film. Additionally, some eye drops may incorporate preservatives to deter bacterial growth. However, prolonged use of preservatives in the ocular area can potentially lead to irritation and inflammation.

Hypochlorous acid is a gentle yet highly effective preservative that can be safely applied to the ocular area. Despite its efficacy, hypochlorous acid is incompatible when paired with various excipients, particularly most demulcents, as it tends to exhibit instability and rapid degradation in solution. This incompatibility has hindered its usage in over-the-counter dry eye formulations. As a result, the prolonged use of alternative preservatives, instead of hypochlorous acid, may potentially lead to undesired side-effects. Consequently, there is a strong need for the development of a stable dry eye formulation that incorporates hypochlorous acid.

### SUMMARY

According to an embodiment, a dry eye composition contains: 50 to 350 ppm of hypochlorous acid, one or more demulcents, and an aqueous vehicle.

In an embodiment of the invention, the composition includes a primary demulcent, polyvinylpyrrolidone having a molecular weight in the range of 15-100 kDa, and preferably in the range of 30-50 kDa. Preferably, polyvinylpyrrolidone is present in the range of 0.02%-10% w/w. In one or more embodiments, the composition further includes a secondary demulcent. The secondary demulcent is selected from the group consisting of propylene glycol, glycerin, sodium carboxymethyl cellulose, PEG 400, PEG 600 and mixtures thereof. The concentration of the second demulcent is less than 1% w/w.

In an embodiment, the aqueous vehicle comprises inorganic salts and/or buffers and organic salts and/or buffers. In an exemplary embodiment, the buffer is an acetate buffer. The pH of the composition is adjusted with the inorganic and organic buffers to be in the range of 3.5 to about 6.5. Additional salts, inorganic and organic, can be included to improve the physiological compatibility of the dry eye composition.

In yet another embodiment, the osmolality of the dry eye composition is in the range of 80 to about 320 mOsm/kg.

In another embodiment, the viscosity of the dry eye composition is in the range of 1 cP to 1000 cP.

According to an embodiment, a kit for managing dry eye comprises the dry eye composition containing hypochlorous acid and polyvinylpyrrolidone; and an applicator. Preferably, the applicator is a dropper device or spray dispenser capable of applying an effective amount of the dry eye composition to the surface of the eye.

In yet another embodiment, a method for managing dry eye involves the application of an effective amount of the dry eye composition disclosed herein to the surface of the eye.

According to an exemplary embodiment, a dry eye composition comprises 50 to 350 ppm hypochlorous acid, 0.02%-10% w/w of 30-50 kDa polyvinylpyrrolidone, and deionized water or USP purified water. The pH of the composition is in the range of 4.0 to 6.5, the osmolality is in the range of 80 to 320 mOsm/kg, and the viscosity is 1-50 cP.

### DETAILED DESCRIPTION

The term and phrases “invention,” “present invention,” “instant invention,” and similar terms and phrases as used herein are non-limiting and are not intended to limit the present subject matter to any single embodiment, but rather encompass all possible embodiments as described.

As used herein, all weight percentages (wt. %) are based on the total wt. % of the dry eye composition, unless otherwise specified. Additionally, all composition percentages are based on totals equal to 100 wt. %, unless otherwise specified.

The compositions and methods for their use can “comprise,” “consist essentially of,” or “consist of” any of the ingredients or steps disclosed throughout the specification. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and can include the ingredients of the present invention and do not exclude other ingredients or elements described herein. The use of the word “a” or “an” when used in conjunction with the term “comprising” in the claims and/or the specification may mean “one,” but it is also consistent with the meaning of “one or more,” “at least one,” and “one or more than one.” As used herein, “consisting essentially of” means that the invention may include ingredients in addition to those recited in the claim, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the composition (as opposed to the degree of utility) is maintained.

All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art.

As used herein, the term “effective amount” of a composition refers to an amount sufficient to elicit the desired biological response. As will be appreciated by those of ordinary skill in this art, and effective amount of a substance may vary depending on such factors as the desired biological endpoint, the patient, etc. The terms effective amount and clinically effect may be used interchangeably herein. For example, an effective amount/clinically effective amount of the composition is the amount that can be used facilitate long term healing and treatment of dry.

While the specification concludes with the claims particularly pointing and distinctly claiming the invention, it is believed that the present invention will be better understood from the following description.

The dry eye composition is formulated to effectively lubricate and protect the eye surface to promote healing. Advantageously, the composition is further configured to prevent the growth and spread of harmful bacteria without irritating the eye and surrounding tissue in the ocular area. The composition contains hypochlorous acid, a demulcent, salts, buffers, and an aqueous vehicle having a physiologically compatible pH and osmolality. Administration of an effective amount of the dry eye composition is accomplished with a dropper device or spray dispenser.

Hypochlorous acid is a weak chlorine oxoacid with a successful history of biological use. The inclusion of hypochlorous acid provides the dry eye composition with antimicrobial activity and enables its use as a preservative agent. The antimicrobial and preservative activity will vary depending on the concentration of hypochlorous acid, the presence of additional excipients, the stability in solution, and the pH of the composition. Maintaining the pH of the composition with buffering agents will further optimize the stability of the hypochlorous acid, its antimicrobial activity, and reduce any potential negative reactions with the eye or skin in the ocular area. The dry eye composition contains at least 50-350 ppm of hypochlorous acid.

Demulcents are agents used in biological compositions to form a protective film over a mucous membrane. Correspondingly, a demulcent applied to the surface of the eye will form barrier that reduces the evaporation of the existing tear film, helps to lubricate the surface, and greatly lessens discomfort for the user. Typical demulcent agents for use on the eye include, but are not limited to, polymers, polyols, dextrans, gelatins, and cellulose derivatives.

Polyvinylpyrrolidone (PVP), commonly called polyvidone or povidone, is a water-soluble synthetic polymer made from the monomer N-vinylpyrrolidone. PVP has many pharmaceutically beneficial properties. Most relevantly, PVP is soluble in aqueous solutions, serves as a demulcent for mucous membranes, and is available in a wide range of molecular weights, which further enables the viscosity of the corresponding solution to be adjusted. Polyvinylpyrrolidone has been used successfully in ophthalmic compositions, including the treatment of dry eye and its symptoms in individuals that both do and do not wear contact lenses.

In an exemplary embodiment, the composition includes a primary demulcent and at least one or more secondary demulcents. The primary demulcent used in the dry eye composition is PVP with a molecular weight in the range of 30-50 kDa and a corresponding viscosity range of 1-50 cP. Importantly, the viscosity of dry eye composition is not limited to be determined solely by the concentration and molecular weight of the PVP demulcent and additional excipients may be employed to adjust the viscosity. The concentration of PVP in the dry eye composition can be in the range of 0.2-10% w/w of the final formulation.

The secondary demulcent can be selected from the group consisting of propylene glycol, glycerin, sodium carboxymethyl cellulose, PEG 400, PEG 600 and mixtures thereof. The concentration of the second demulcent is less than 1% w/w of the final formulation.

The aqueous vehicle can be deionized water or USP purified water and can incorporate various components for the purpose of adjusting viscosity, osmolality, and pH as necessary. The osmolality of the solution can be adjusted to be within the range of approximately 80 to 320 mOsm/kg. Ideally, the pH of the solution is maintained within the range of 3.5 to 6.5. To adjust the pH, inorganic buffers such as phosphate salts or organic buffers like acetates, citrate and tris salts, along with their corresponding acids or bases, can be added in specific ratios during the preparation of the aqueous vehicle. In an exemplary embodiment, a preferred buffer is an acetate buffer. Alternatively, the pH can be adjusted by gradually introducing small amounts of an acid or a base while stirring until the desired pH is attained, once all the ingredients have dissolved or dispersed. Furthermore, the aqueous vehicle can also include additional inorganic salts and ions to ensure it is compatible with the physiological conditions of the eye, making it suitable for use as a dry eye solution.

According to an embodiment, a kit for managing dry eye comprises (i) the dry eye composition containing hypochlorous acid and polyvinylpyrrolidone; and (ii) an applicator. Preferably, the applicator is a dropper device or spray dispenser capable of applying an effective amount of the dry eye composition to the surface of the eye.

In yet another embodiment, a method for managing dry eye involves the application of an effective amount of the dry eye composition disclosed herein to the surface of the eye.

For example, the dry eye composition is typically applied to the surface of the eye in the form of a drop or a spray. In certain embodiments, the composition may be applied for a duration/frequency prescribed by a physician. The eyelid cleanser composition can be contained and dispensed from a suitable applicator. According to one or more embodiments, the final dry eye composition is sterile filtered into high density polyethylene, low density polyethylene, or polypropylene dropper bottles, glass or plastic bottles with an auxiliary dropper device, or a spray dispenser.

### Experimental Data: Stability of Hypochlorous Acid in the Presence of Excipients

TABLE 1
Stability of hypochlorous acid (HOCl) in presence of
excipients present at 1% w/w (other than PVP)

| | | FAC (ppm) | | | | | |
|---|---|---|---|---|---|---|---|
| Sample | Temperature | 0 d | 3 d | 4 d | 5 d | 6 d | 7 d |
| HOCl-HA | 25° C. | 240 | 94 | 131 | 105 | 86 | 83 |
| | 40° C. | 240 | 37 | 20 | 23 | N/D | N/D |
| HOCl-CMC | 25° C. | 240 | 152 | 184 | 155 | 172 | 186 |
| | 40° C. | 240 | 120 | 126 | 65 | 21 | 9 |
| HOCl-HPMC | 25° C. | 240 | 12 | 5 | N/D | N/D | N/D |
| | 40° C. | 240 | 13 | 6 | N/D | N/D | N/D |
| HOCl-Poly 80 | 25° C. | 240 | 97 | 59 | 88 | 80 | 70 |
| | 40° C. | 240 | 38 | 18 | 17 | 8 | 9 |
| HOCl-PG | 25° C. | 240 | 165 | 218 | 219 | 171 | 199 |
| | 40° C. | 240 | 141 | 145 | 116 | 102 | 61 |
| HOCl-Glycerin | 25° C. | 240 | 222 | 223 | 194 | 210 | 111 |
| | 40° C. | 240 | 139 | 152 | 128 | 66 | 92 |
| HOCl-PEG400 | 25° C. | 240 | 183 | 205 | 199 | 190 | 156 |
| | 40° C. | 240 | 156 | 145 | 117 | 139 | 110 |
| HOCl-Alginate | 25° C. | 240 | 174 | 185 | 130 | 151 | 103 |
| | 40° C. | 240 | 72 | 62 | 48 | 32 | 40 |

HA = hyaluronic acid (sodium salt);
CMC = carboxymethyl cellulose (CMC, sodium salt),;
HPMC = Hypromellose;
PG = propylene Glycol;
Poly 80 = polysorbate 80;
N/D = none detected

TABLE 2
Stability of hypochlorous acid (HOCl) in
presence of PVP (2% w/w)

| | | FAC (ppm) | | | | | |
|---|---|---|---|---|---|---|---|
| Sample | Temperature | 0 d | 6 d | 13 d | 34 d | 69 d | 97 d |
| HOCl- | 25° C. | 319.61 | Not measured | Not measured | 258.47 | 228.17 | 221.64 |
| PVP | 40° C. | 319.61 | 252.0333 | 236.69 | 209.19 | 138.17 | 114.7 |

The results in Tables 1 and 2 indicate that hypochlorous acid has increased stability in the presence of PVP versus other commonly used excipients and demulcents. For example, after 34 days at 40° C. the FAC concentration, relative to the starting concentration, is greater in the presence of PVP than all other excipients at 40° C. after 7 days. Thus, the present invention and its embodiments, as described herein, incorporate hypochlorous acid and a demulcent with exceptional stability in a dry eye formulation.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

1. A dry eye composition consisting of:
(a) hypochlorous acid; and
(b) one or more demulcents, wherein at least one of the demulcents is polyvinylpyrrolidone, and
wherein additional demulcents are less than 1% w/w, wherein the additional demulcents are selected from the group consisting of propylene glycol glycerin, sodium carboxymethyl cellulose, PEG 400, PEG 600 and mixtures thereof.
(c) deionized or purified water, and
(d) inorganic and organic salts and/or buffers, wherein the pH of the composition is in the range of 3.5-6.5.

2. The dry eye composition according to claim 1, wherein the concentration of the hypochlorous acid is in the range of 50 to 350 ppm.

3. The dry eye composition according to claim 1, wherein the concentration of polyvinylpyrrolidone is in the range of 0.02%-10% w/w.

4. (canceled)

5. The dry eye composition according to claim 1, wherein the molecular weight of polyvinylpyrrolidone is 15-100 kDa.

6. (canceled)

7. (canceled)

8. The dry eye composition according to claim 1, wherein the buffer is selected from the group consisting of an organic and an inorganic buffer buffer.

9. (canceled)

10. (canceled)

11. (canceled)

12. The dry eye composition according to claim 1, wherein the osmolality is in the range of 80-320 mOsm/kg.

13. The dry eye composition according to claim 1, wherein the viscosity is in the range of 1-50 cP.

14. A kit for managing dry eye, wherein the kit comprises:
the eye composition according to claim 1; and
an applicator for the dry eye composition.

15. A kit for managing dry eye according to claim 14, wherein the applicator is an integrated dropper device, a bottle with an auxiliary dropper device, or a spray dispenser.

16. A method for managing dry eye, the method comprising the application of an effective amount of the dry eye composition according to claim 1 to the eye.
